(19) United States
(12) Patent Application Publication
(10) Pub. No.: US 2023/0226056 A1
(43) Pub. Date: Jul. 20, 2023

(54) COMPATIBLE SOLUTES FOR PREVENTING OR TREATING SARS-COV-2 INFECTIONS

(71) Applicant: bitop AG, Dortmund (DE)

(72) Inventors: Thomas SCHWARZ, Koeln (DE); Eva GALIK, Muenchen (DE); Dieter OESTERHELT, Muenchen (DE)

(73) Assignee: bitop AG, Dortmund (DE)

(21) Appl. No.: 17/924,565

(22) PCT Filed: May 10, 2021
(86) PCT No.: PCT/EP2021/062293

§ 371 Date: Nov. 10, 2022

(30) Foreign Application Priority Data
May 10, 2020 (DE) 102020112603.4

(51) Int. Cl. A61K 31/505; A61K 31/7032; A61K 31/205; A61K 31/4164; A61K 38/05; A61K 38/06; A61K 9/00; A61P 31/14; G01N 33/50

(52) U.S. Cl. CPC A61K 31/505; A61K 31/7032; A61K 31/205; A61K 31/4164; A61K 38/05; A61K 38/06; A61K 9/0078; A61P 31/14; G01N 33/5008

(57) ABSTRACT

The present invention relates to the use of organic and highly water-soluble compatible solutes or a solute mixture, preferably in the form of an inhalable, oropharyngeally, nasally and intravenously administrable composition, in the prevention or treatment of diseases caused by ss(+)RNA viruses of the Coronavriridae family, preferably of those diseases caused by SARS-CoV-1, SARS-CoV-2, MERS-CoV, HCoV-HKU1, HCoV-OC43, HCoV-NL63 and/or HCoV-229E. Particularly suitable solutes in the meaning of the invention are ectoine and its derivatives, Glycoin, mannosylglycerate (Firoin) and mannosylglyceramide (Firoin-A), which, due to their strong water-binding capacity, reduce the binding of the viruses to the receptors of the host cell in the transitional epithelium, e.g. eye, in the internal epithelium, e.g. lung, and in the endothelium and thus reduce or prevent the multiplication of the viruses. According to the invention, prevention is enabled by a reduced infectious sputum and breath, and treatment and rehabilitation of the affected tissues is enabled by the membrane protective properties of the compatible solutes according to the invention.

The present invention relates to the use of organic and highly water-soluble compatible solutes or a solute mixture, preferably in the form of an inhalable, oropharyngeally, nasally and intravenously administrable composition, in the prevention or treatment of diseases caused by ss(+)RNA viruses of the Coronavriridae family, preferably of those diseases caused by SARSCoV-1, SARS-CoV-2, MERS-CoV, HCoV-HKLH, HCoV-OC43, HCoV-NL63 and/or HCoV-229E.1.

Particularly suitable solutes in the meaning of the invention are ectoine and its derivatives, glycoin, mannosylglycerate (Firoin) and mannosylglyceramide (Firoin-A), which, due to their strong water-binding capacity, reduce the binding of the viruses to the receptors of the host cell in the transitional epithelium, e.g. eye, in the internal epithelium, e.g. lung, and in the endothelium and thus reduce or prevent the multiplication of the viruses. According to the invention, prevention is enabled by a reduced infectious sputum and breath, and treatment and rehabilitation of the affected tissues is enabled by the membrane protective properties of the compatible solutes according to the invention.

The new SARS coronavirus 2 (SARS-CoV-2) has quickly become a global challenge. Within a very short time, the spread was declared a pandemic. Although the course of the disease is mild in many to most cases and only slight symptoms such as malaise, fever and possibly coughing can progress to acute respiratory distress syndrome (ARDS) and severe acute respiratory syndrome (SARS). The mortality rate increases with the severity of the disease and can reach as high as 49% in critically ill patients. Currently, there is no targeted treatment for the Covid-19 diseases caused by the Sars-CoV-2 virus, synonymous with “Coronavirus Disease 2019”. Currently, only supportive measures can be applied, since no effective therapeutic agent against the Covid-19 diseases and no vaccination against the SARS-CoV-2 virus are available at this time.

Against this background, the present invention is based on the task of providing a compound, an agent, a medical product and/or a pharmaceutical for the prevention and/or treatment of diseases which are caused by ss(+)RNA viruses of the Coronavriridae family, in particular viral infections and/or inflammations caused by the virus SARS-CoV-1, SARS-CoV-2, MERS-CoV, HCoV-HKU1, HCoV-OC43, HCoV-NL63 and/or HCoV-229E.

The task is to prevent the penetration of the aforementioned viruses, in particular SARS-CoV-1, SARS-CoV-2 and/or MERS-CoV, into a host cell, which host cells are human and animal eukaryotic cells. The aim is to provide a compound, agent, medical device and/or drug for use in the prevention and treatment of the aforementioned diseases in humans and animals. In order to identify such compounds, the present invention has the further object of providing a method which identifies precisely those compounds which are suitable for the treatment and prevention of the aforementioned diseases. A further object of the present invention is the provision of suitable medical products for individual prevention for daily use and an isolated and complementary therapy for the treatment of damaged epithelial tissue and the endothelium. Therefore, it is a further task to provide suitable formulations and dosage forms.

Surprisingly, it has now been found that compatible solutes such as ectoine and its derivatives glycoin, mannosylglycerate (Firoin) or mannosylglyceramide (Firoin-A) can solve the above tasks, as shown in the attached examples and FIG. 3-5.

Therefore, an object of the present invention is a compatible solute or solute mixture for use in the prevention or treatment of diseases caused by ss(+)RNA viruses of the Coronavriridae family, wherein the at least one compatible solute is selected from organic and highly water soluble, preferably bio-based, compounds, preferably selected from hydroxyectoine and ectoine and the derivatives. If “ectoine and/or the derivatives” or “ectoine and/or its derivatives” are mentioned in the present text, all compounds of the formula I and II are included.

The general classification of viruses is known to those skilled in the art. To classify the viruses of the family Coronaviridae considered by the present invention, these are distinguished from the viruses of the family Picornaviridae (order Picornavirales), the family Adenoviridae (suborder unknown) and the family Filoviridae (order Mononegavirales). Viruses of the Picornaviridae family include rhinoviruses which have positive polarity single-stranded RNA genomes. Viruses of the Adenoviridae family include adenoviruses based on ds-DNA and viruses of the Filoviridae family, which include e.g. Ebola virus, are also single-stranded RNA genome but with negative polarity. Viruses within the meaning of this invention belong to the family Coronaviridae, which is divided into two subfamilies Coronavirinae and Torovirinae. Coronavirinae are divided into the genera alpha, beta, which only infect mammals, gamma, and delta, which infect both mammals and birds.

E229 and NL63 are human pathogenic alphacoronaviruses, while OC43 and HKU1 and all new CoVs viruses comprising SARS-CoV2 belong to the betacoronavirus genus. Therefore, another subject of the present invention is the use of at least one compatible solute or solute mixture, preferably ectoine and its derivatives, in which the disease is caused by an ss(+)RNA virus of the Betacoronavirus and/or Alphacoronavirus genus, and preferably by a virus selected from SARS-CoV-1, SARS-CoV-2, MERS-CoV, HCoV-HKU1, HCoV-OC43, HCoV-NL63 and/or HCoV-229E.

The four viruses HCoV-HKIM, HCoV-OC43, HCoV-NL63 and HCoV-229E cause rhinitis, conjunctivitis, pharyngitis, occasionally laryngitis and/or otitis media and thus mainly diseases of the upper respiratory tract. In contrast, the other viruses can cause more serious lower respiratory tract diseases with or without systemic infections and/or inflammation. Therefore within the meaning of the invention, particularly preferred viruses are ARS-CoV(-1), SARS-CoV-2 and/or MERS-CoV.

Each of the above viruses comprises a surface protein bound in the viral envelope which interacts with specific surface proteins of the host cells, binds to them, ultimately inducing infection of the host cell (Tay et al.). Therefore, in a particular embodiment of the present invention, at least one compatible solute or solute mixture, preferably ectoine, is used, in which the ss(+)RNA virus interacts with at least one membrane-bound protein or component thereof on human cells (host cells) and uses this protein or the component thereof as a receptor to bind to the cell. According to the invention, the at least one solute acts on all surface proteins of human cells which are bound by viral pathogens as receptors through pathogenic surface proteins. In a particular embodiment, the ss(+)RNA virus interacts with a receptor selected from angiotensin converting enzyme 2 (ACE2), aminopeptidase N (APN) and/or dipeptidyl peptidase 4 (DPP4). These viral receptors interact with various proteins of the viruses of the invention, as shown in FIG. 1 of Fang Li 2020. In particular, carboxypeptidases and aminopeptidases and dipeptidyl peptidases other than those already mentioned are also covered by the present invention. “Viral receptors” within the meaning of the invention are all membrane-bound proteins which are recognized by the ss(+)RNA viruses mentioned here as receptors on their host cells, preferably on human cells, particularly preferably on cells of the transitional epithelial tissue, the internal epithelial tissue and/or or the endothelium.

Therefore, in a particular embodiment of the method below for identifying potential compatible solutes according to the invention, the corresponding combinations are tested according to Fang Li 2016 in order to identify in this way the most suitable compatible solute for the respective virus, which is selected from organic and highly water-soluble, preferably bio-based, compounds, preferably hydroxyectoine and ectoine and the derivatives. These solutes particularly preferably have a water-binding capacity of greater than or equal to 7 mol/mol H₂O/solute.

A further object of the present invention is namely a method for identifying a compatible solute according to the invention for use in the prevention or treatment of diseases caused by ss(+)RNA viruses of the Coronavriridae family, wherein the at least one compatible solute is selected from organic and highly water-soluble, preferably bio-based, compounds. The method (synonym: biological assay) comprises the following steps:
providing a cell line which has membrane-bound surface proteins as potentially viral receptors, preferably a cell line from table 5,
contacting the cells with a compound which is potentially a compatible solute within the meaning of the invention, preferably ectoine and/or another compound of formula I and/or II, glyceryl glucoside (Glycoin), mannosylglycerate (Firoin), mannosylglyceramide (Firoin-A),
preferably a control approach without any of the potential solutes,
adding a viral receptor binding domain comprising a measurable signal, preferably an angiotensin converting enzyme 2 (ACE2), aminopeptidase N (APN) and/or dipeptidyl peptidase 4 (DPP4), preferably the S1 protein or another according to Fang Li 2016,
incubation of the approach, preferably for a sufficient time for the interaction and binding of the binding partners
recording the signal measurable on the cell, preferably a fluorescence signal, and
determination of a reduced binding between the viral receptor binding domain and the human membrane-bound surface protein.

If a reduction in the signal emanating from the viral protein is measured in the above-mentioned method compared to the control carried out, this indicates reduced binding of the virus. Example 1 demonstrates the functionality of the assay. In the method according to the invention, the cells are preferably incubated with propidium iodide, so that membrane-damaged, preferably dead cells, are subtracted from the measurement signal. The potential compounds are selected from organic and highly water-soluble, preferably bio-based, compounds that preferably have a water-binding capacity of greater than or equal to 7 mol/mol H₂O/solute.

Compatible solutes according to the invention are preferably screened using the above method. The method according to the invention can be configured in two different embodiments. In a first alternative embodiment, compatible solutes with a water-binding capacity of greater than or equal to 7 mol/mol H₂O/solute are selected in an upstream process, measured in particular by atomic force spectroscopy according to Rouychoudhury et al 2011, and then fed to the biological assay described above. In a second alternative of an embodiment of the method according to the invention, potential solutes are first identified in the aforementioned biological assay and then the water-binding capacity is determined according to Rouychoudhury et al 2011. In a further embodiment of the method and of the alternatives, ectoine is carried along as an internal standard to identify compatible solutes according to the invention measured in terms of ectoine.

A compatible solute according to the invention, preferably according to formula I and/or II, can have biological (bio-based) origin or can also be produced synthetically. Bio-based compatible solutes, preferably compounds of formula I and/or II, can be produced biotechnologically using natural strains (see also Costa et al), e.g. Halomonas elongata et al. (Table 1), or using genetically modified microorganisms, e.g. Corynebacterium glutamicum. The use of genetically modified microorganisms enables the production of larger quantities of compatible solutes according to the invention. Likewise, the synthetic production of compatible solutes according to the invention is advantageous in terms of quantity and costs.

Regardless of the production route, however, the water-binding capacity and the reducing/interfering effect of the respective compatible solute on the binding between ss(+)RNA viruses and host cells are essential for the purposes of the invention, preferably on the binding between the viral peplomers or spike proteins, preferably the receptor binding domains (RBD), and the viral receptors on the host cell, preferably ACE2, APN and/or DPP4 (see above). Compatible solutes according to the invention have a water-binding capacity of greater than or equal to 7 mol/mol H₂O/solute, preferably determined according to Rouychoudhury et al 2011.

Synthetically produced compatible solutes, such as ectoine, and ectoine produced by biosynthesis differ in several characteristics of the end product. Features include the purity of the end product in relation to residues of the chemicals used for the synthetic production, the purity in relation to the enantiomeric purity, smell, color (Hazen number) and processing into a composition according to the invention. A comparative example of a commercially synthetically produced ectoine and a bio-based ectoine shows the following differences:

| | biotechnical | Synthetic |
|---|---|---|
| | ectoine | ectoine |
| Amount of ectoine [%] | 98.68 | 96.89 |
| Hazen number (2%) | 2 | 1 |
| Optical rotation | 141 | different |
| pH (2%) | 6.76 | 6.62 |
| Hydroxyectoin [%] | 1.33 | n/a |
| DABAs | n/a | n/a |
| 2,4-diaminobytteric | | |
| acid | | |
| Other abnormalities | none | “Synthetic” odor; |
| | | insoluble residue |
| | | in the ectoine |
| | | solution for |
| | | methanol |
| | | determination. |

Ectoine fall within the formulas I and II and can exist as optical isomers, diastereomers, racemates, dipolar ion, cations or as a mixture of at least two of the aforementioned forms. Isomers include (R,R)-, (R,S)-, (S,S)- and (S,R)-configurations of the compounds of formula I and formula II, wherein an S-enantiomer according to the Fischer projection corresponds to the L-enantiomer and one R-enantiomer corresponds to the D-enantiomer e.g. L-ectoine equals S-ectoine, D-ectoine equals R-ectoine.

In a preferred embodiment of the solute or solute mixture according to the invention, the at least one compatible solute is present in enantiomerically pure form with a purity of greater than or equal to 90%, preferably greater than or equal to 95%, greater than or equal to 97%, greater than or equal to 99%, particularly preferably equal to 100%. Based on the solute mixture, this means that the mixture of two compounds has the respective compounds in enantiomerically pure form and preferably has no contamination of the selected compounds by the isomer.

Enantiomerically pure forms of the solute or solute mixture according to the invention particularly preferably have S and/or (S,S) isomers. In a preferred enantiomerically pure solute mixture, S-ectoine and (S,S)-hydroxyectoine are each present with a purity of greater than or equal to 90%, greater than or equal to 95%, preferably greater than or equal to 97%, greater than or equal to 99%, particularly preferably equal to 100%. This solute mixture therefore preferably has less than or equal to 10% less than or equal to 5%, preferably less than or equal to 3%, less than or equal to 1%, particularly preferably equal to 0% of R-ectoine or (R,S)-/(S,R)- or (R,R)-hydroxyectoine.

In a particular embodiment of the invention, the following racemates are preferred:
S-ectoine and R-ectoine,
(S,S)-Hydroxyectoine, (S,R)-Hydroxyectoine, (R,S)-Hydroxyectoine and (R,R)-Hydroxyectoine, or
S-Homoectoine and R-Homoectoine.

In addition to isomers, diastereomers, racemates, dipolar ion, cations and mixtures of the aforementioned compounds are also the subject of the invention. Derivatizations can be carried out with hydroxy, sulfonic acid, carboxy acid derivatives such as amides, esters etc., carbonyl, ether, alkoxy and dydroxyl groups.

In a particular embodiment of the use according to the invention, at least one compatible solute is selected from glyceryl glucoside (Glycoin), glycine betaine, mannosylglycerate (Firoin), mannosylglyceramide (Firoin-A), ectoine and its derivatives of the formula I and/or II and the physiologically compatible salts, amides and esters of the aforementioned compounds, where in formula I and in formula II

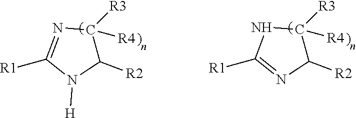

R1=H or Alkyl,

R2=H, COOH, COO-Alkyl or CO—NH—R5,

R3 and R4 are each independently H or OH,

R5=H, Alkyl, an amino acid residue, dipeptide residue or tripeptide residue

n=1, 2 or 3,

Alkyl=an alkyl residue with C₁-C₄ Carbon atoms

Also preferred compatible solutes according to the invention are compounds from the group comprising ectoine and derivatives thereof, glycoin (glyceryl glucoside), L-proline, mannosylglycerate, N-acetyldiaminobutyric acid (NADA), trimethylamine N-oxide (TMAO) and/or glycine betaines. Preferred derivatives of ectoine include S/R-ectoine, (S,S)/(R,R)-hydroxyectoine, (S,S)/(R,R)-hydroxyectoine and S-homoectoine, and the physiologically compatible salts, amides and esters of the aforementioned compounds.

According to the invention, a solute mixture of at least two of the aforementioned compounds is also used, preferably a solute mixture comprising at least two compounds of formula I and/or formula II. The respective compatible solute of formula I or II is preferably in enantiomerically pure form with a Purity greater than or equal to 90%. A particularly preferred solute mixture within the meaning of the invention of compounds of the formula I or II contains, based on the sum of all compounds with a total content of 100% by weight, greater than or equal to 85% by weight of S-ectoine and less than or equal to 15% by weight (S,S)-Hydroxyectoine.

In a particular embodiment, compatible solutes according to the invention are bio-based, bio-based meaning that the compound has a biological origin. Biological sources of solutes according to the invention include e.g. from algae, fungi, phototrophic bacteria, methanogenic bacteria, Actinopolyspora halophila, gamma proteobacteria, Nocardiopsis sp., Brevibacteria, gram-positive cocci, many bacilli, algae, some bacilli and related species (Planococcus citreus), Staphylococcus epidermidis, Salinicoccus sp., strain M96/12b, Sporosarcina halophila, methanogenic bacteria (R-glutamine, Ne-acetyl-β-lysine), Ectothiorhodospira marismortui (CGA), other anoxigenic phototrophic bacteria, Azospirillum brasilense, Rhizobium meliloti and many more. Some examples are summarized in table 1 below.

A further summary of compatible solutes within the meaning of the invention is contained in Costa et al 1998. The compounds listed there from page 122 to page 141 are hereby incorporated as part of the present invention. The solutes mentioned there are suitable for use according to the invention, preferably those with a water-binding capacity of greater than or equal to 7 mol/mol H₂O/solute, preferably determined according to Rouychoudhury et al 2011.

TABLE 1
Examples of compatible Solutes

| | | |
|---|---|---|
| Solut | Abbreviation | Origin |

Polyole

| | | |
|---|---|---|
| Arabitol, Arabinitol | | Saccharomyces rouxii, Asteromyces |
| | | cruciatus |
| Dimannosyl-di-myo-inositol- | DMIP | Thermococcus spp., Thermotoga |
| 1,1′(3,3′)-phosphat | | maritima, Thermotoga neapolitana |
| Di-myo-Inositol-1,1′-phosphat | DIP | Pyrococcus woesei, Methanococcus |
| | | igneus, Pyrococcus furio-sus, |
| | | Thermococcus litoralis |
| Diglycerinphosphat | DGP | Archaeoglobus fulgidus |

Sugars

| | | |
|---|---|---|
| Firoin A, Mannosylglyceramid, | MGA | Rhodotermus marinus |
| Mannosylglycerinsäureamid | | |
| Firoin, Mannosylglycerat, | MG | Rhodotermus marinus, Pyrococcus |
| Mannosylglycerinsäure | | furiosus, Pyrococcus horikoshii, |
| | | Dehalococcoides ethenogenes, Thermus |
| | | thermophilus, Rubrobacter xylanophilus, |
| | | Petrotoga miotherma, Palaeococ-cus |
| | | ferrophilus, Petrotoga miotherma |
| Glyceryl Glucoside | Glycoin | Myrothamnus flabellifolia, |
| | | Cyanobakterien, Mikroalge Spirulina. |
| Trehalose, Mykose | | Plants, fungi, insects, bacteria, Thermus |
| | | thermophilus |

Amino acids

| | | |
|---|---|---|
| Dimethylpropionat, Pivalinsäure | | Plants, fungi, |
| (S)-2-Methyl-3,4,5,6-tetra- | S-Ectoine | Ectothiorhodospira halochloris, |
| hydropyrimidin-4-carbonsäure | | Halomonas elongata, Marinococcus |
| | | halophilus, Brevibacterium linens, |
| | | Halomonas SPC1, Volcaniella |
| | | eurihalina, Deleya Salina, Bacillus |
| | | pantothenticus, Bacillus halophilus, |
| | | Salibacillus salexigens Vibrio costicola |
| | | und Streptomyces parvulust |
| (4S,5S)-5-Hydroxy-2-methyl- | Hydroxyectoine | |
| 1,4,5,6-tetra-hydropyrimidin-4- | | |
| carbonsäure | | |
| γ-NAc-DABA, γ-N-Acetyl-2,4-di- | NADA | Ectoine synthesis |
| aminobuttersäure, 2(S)-4-Acet- | | |
| amido-2-aminobutansäure, 2(S)- | | |
| 4-Acetamido-2-aminobuttersäu-re, | | |
| γ-N-Acetyl-2,4-diaminobutan- | | |
| säure, γ-N-Acetyl-2,4-diaminobut- | | |
| tersäure, γ-N-Acetyldiamino- | | |
| butansäure, γ-N-Acetyldiamino- | | |
| buttersäure, N-Acetyldiamino- | | |
| buttersäure | | |
| β-Galactopyranosyl-5- | GAL-HYL | Thermococcus litoralis, Archaea |
| hydroxylysin | | |
| α-NAc-DABA, α-N-Acetyl-2,4-di- | | Euphorbia pulcherrima |
| aminobuttersäure, 2(S)-2-Acet- | | |
| amido-4-aminobutansäure, 2(S)- | | |
| 2-Acetamido-4-aminobuttersäu-re, | | |
| α-N-Acetyl-2,4- | | |
| diaminobutansäure, α-N-Acetyl- | | |
| 2,4-diamino-buttersäure, α-N- | | |
| Acetyldiamino-butansäure, α-N- | | |
| Acetyldiamino-buttersäure, N- | | |
| Acetyldiamino-buttersäure | | |

Therefore, compatible solutes within the meaning of the invention are compounds from the classes of sugars, amino acids and polyols, which are characterized by OH groups and/or amino and/or amide groups, which have a high reactivity with water. Also included are betaines, compounds of formula I/II, proline, carboxamides, NAc—O, NAc-L and mannosylglycerate.

Thus, a compatible solute according to the invention (synonym: solute) is an organic and highly water-soluble, preferably bio-based, compound which, in a particular embodiment, has a water-binding capacity of greater than or equal to 7 mol/mol H₂O/solute. The water binding capacity is preferably greater than or equal to 7.2, greater than or equal to 7.5, greater than or equal to 7.7, greater than or equal to 8.0, greater than or equal to 8.2, greater than or equal to 8.5, greater than or equal to 8.7, greater than or equal to 9, respectively as [mol/mol H₂O/solute]. The preferred solute for the purposes of the invention is ectoine with a water-binding capacity of greater than or equal to 9.0 mol/mol H₂O/solute.

The respective preferred solute or solute mixture within the meaning of the invention can be used alone or in combination with other physiological solutions, e.g. various infusion solutions used in the clinic, NaCl solutions.

The solutes of the invention, preferred ectoine and/or its derivatives, or a composition containing the at least one preferred solute, are used in the prevention or treatment of viral diseases involving infection and/or inflammation of the transitional epithelial tissues and/or internal epithelial tissues. In a particular embodiment of the use according to the invention, the viral disease comprises an infection and/or inflammation of the endothelium.

Viral disease within the meaning of the invention include an infection and/or inflammation of the endothelium, in particular the endothelium of the eye, the upper and/or lower respiratory tract, the trachea, the lungs, the bronchi, the bronchial tree, the heart, the endothelium of heart, blood-lymphatic vessels, cerebral vessels, renal vessels, esophagus, gastric mucosa and/or small/intestinal mucosa.

Therefore, within the meaning of the invention, such viral diseases are included, each caused by SARS-CoV-1, SARS-CoV-2, MERS-CoV, HCoV-HKU1, HCoV-OC43, HCoV-NL63 and/or HCoV-229E, which show an infection and/or inflammation of the upper and/or lower airways, trachea, lungs, bronchi and/or bronchial tree. These diseases are characterized and detectable in that the tissues affected in the aforementioned organs have been infected by the virus and thus at least one component (viral RNA) of the virus can be detected in these tissues. Affected tissues within the meaning of the invention include
transitional epithelial tissues including upper airways, oral cavity, oral mucosa, gums, tongue, lingual mucosa, nasal cavity, paranasal sinuses, nasal mucosa, eye, vocal folds, pharynx and genitals and/or
internal epithelial tissues including lower airways, trachea, bronchi, bronchial tree, lungs, internal endothelial tissue, continuous endothelium, in particular continuous endothelium of lungs and heart, endothelium of heart, blood, lymph vessels, esophagus, gastric mucosa and/or small/intestinal mucosa.

TABLE 2
Epithelial tissue within the meaning of the invention

| Epithelial | |
|---|---|
| tissue | appearance |
| Single layer | Serous membranes comprising pleura, pleura |
| squamous | of lung, pericardium, vaginal membrane of |
| epithelium | testis; alveolar epithelium, endothelium, lung |
| | (endothelium), endothelium of heart, blood |
| | and lymph vessels; mucous membrane of |
| | tongue (underside of tongue). |
| Single layer | Epithelium of stomach, small and large |
| high prismatic | intestine; gastric mucosa, intestinal mucosa |
| epithelium | |
| Double row | Salivary gland, oral cavity, lacrimal duct |
| epithelium | |
| Multi-row | Nasal cavity, pharynx (throat), larynx, |
| high prismatic | airways, bronchial tree, Eustachian tube, |
| epithelium | urethra, |
| Multilayer non | Anterior corneal epithelium (eye), vocal fold, |
| keratinised | oral cavity, gums (internal marginal epithelium |
| epithelium | surrounding the neck of the tooth), pharynx, |
| | esophagus, anus, vagina, mucous membrane |
| | of the tongue (upper side of the tongue); |
| | conjunctiva of the eye, cornea. |
| Multilayer | Epidermis (stratum basale, stratum spinosum, |
| keratinized | stratum granulosum), nasal vestibule, external |
| epithelium | auditory canal, gums (external marginal |
| | epithelium to the oral cavity), |

Treatment and prevention within the meaning of the invention is to be interpreted broadly and also includes support for the rehabilitation of tissue damaged by the virus. This is particularly relevant when immunization by vaccines is the priority and the tissue damaged by the virus is to be rehabilitated by a combination according to the invention by using a solute according to the invention, or the tissue is to be protected preventively.

In a particular embodiment of the use according to the invention, the at least one solute or solute mixture, preferably in the form of a composition according to the invention, is used in the prevention or treatment of respiratory diseases caused by the aforementioned viruses, preferably by SARS-Cov-2. Compositions according to the invention contain at least one compatible solute selected from organic and highly water-soluble, preferably bio-based, compounds, preferably from hydroxyectoine and ectoine and the derivatives, and preferably have a water-binding capacity of greater than or equal to 7 mol/mol H₂O/solute.

Viral diseases of the airways within the meaning of the invention include not only endothelial infections and/or inflammations, but also infections and/or inflammations of the alveolar epithelial cells. Viral diseases within the meaning of the invention, which are caused by ss(+) RNA viruses, preferably by SARS-Cov-2, include pneumonia, SARS (Severe Acute Respiratory Syndrome) and organ-wide damage to the aforementioned tissue. SARS-Cov-2 or COVID-19 patients show typical symptoms such as fever, malaise, tiredness and cough. Most adults or children infected with SARS-CoV-2 have mild flu-like symptoms. These can quickly progress to acute respiratory distress syndrome (SARS), respiratory failure, multiple organ failure, and even death in some patients, particularly those in high-risk groups.

Other symptoms include cough, sputum production, shortness of breath, sore throat and headache. Some patients present with gastrointestinal symptoms including diarrhea and vomiting. Fever and cough are the dominant symptoms, while upper respiratory and gastrointestinal symptoms were rare.

The products according to the invention are particularly suitable for the prevention or treatment of patients with the aforementioned symptoms of the upper and/or lower respiratory tract. Particularly preferred are inhalable compositions and products (FIG. 6). The products according to the invention (FIG. 6) which contain at least one compatible solute which is selected from organic and highly water-soluble, preferably bio-based, compounds, preferably hydroxyectoine, ectoine and/or the derivatives. These compatible solutes particularly preferably have a water-binding capacity of greater than or equal to 7 mol/mol H₂O/solute. These products fulfill two functions. On the one hand they support the rehabilitation of the attacked tissue (see table 2) and in this way treat the viral disease within the meaning of the invention. On the other hand, the environment is protected because less infectious sputum and breath is achieved.

A particular embodiment of the use according to the invention is the use of the at least one compatible solute, preferably ectoine and/or its derivatives, in the treatment or prevention of systemic endotheliitis as caused by SARS-Cov-2. The so-called Covid-19 endotheliitis is characterized by multiple organ damage and in particular by diffuse endothelial inflammation in the heart, liver and kidneys. Cardiovascular problems are also described. In these patients, it is found that SARS-CoV-2 directly triggers inflammation in the vessels. Other organs may show infection and/or inflammation.

In a particular embodiment of the use according to the invention of the at least one compatible solute or solute mixture, preferably contained in a composition according to the invention, the at least one compatible solute, preferably ectoine, reduces or prevents the unfolding and/or opening of the viral protein suitable for binding to the human receptor of the ss(+)RNA virus. The viral proteins, also called peplomers, of the ss(+)RNA viruses according to the invention include the so-called S1 spike proteins of the viruses according to the invention and in particular the respective binding domains comprising domain A of HCoV-OC43 S, domain B of SARS-CoV S, which interacts with the ACE2 receptor, HCoV-NL63 S, MERS-CoV S and HCoV-229E S (Tortoricia 2019). The interfering effect of the solutes of the invention on the interaction and binding between the peplomers and the viral receptors is shown in FIGS. 3 and 4.

The viruses have the peplomers (spike proteins) on the viral envelope, the peplomer comprising a glycosylated S-protein (spikes protein, 180-220 kDa) forming a membrane-anchored trimer. These parts carry both (S1) the receptor binding domain (RBD), with which the virus can dock to a cell, and (S2) a subunit that, as a fusion protein (FP), causes the virus envelope and cell membrane to fuse. The receptor binding domain (RBD) includes an N-terminal domain (NTD) and a C-terminal domain (CTD), both of which can function as a receptor binding domain and bind different proteins and sugars.

The SARS-CoV-2 spike protein (S protein) plays a central role in viral infection and pathogenesis. S1 recognizes and binds to host receptors, and subsequent conformational changes in S2 facilitate fusion between the viral envelope and the host cell membrane. The mechanism of infection involves the steps of binding of the viral receptor-binding domain to the host cell receptor, followed by fusion of the membranes, entry of the viral RNA into the host cell, propagation of the viral RNA using the cellular machinery of the host cell, and Exit of the virus from the host cell.

In a preferred embodiment of the use according to the invention, the at least one compatible solute or solute mixture, preferably ectoine and/or its derivatives, preferably contained in a composition according to the invention, reduces or prevents the fusion of the viral membranes with the membrane of the attacked cell, in particular an epithelial cell and/or or endothelial cell. In a particular embodiment of the use according to the invention, the multiplication of the ss(+)RNA virus, in particular in the host cell, is reduced by the at least one compatible solute, preferably ectoine and/or its derivatives, preferably biobased, preferably contained in a composition according to the invention or prevented.

The surprising effect of compatible solutes according to the invention is shown in example 3 and in FIGS. 4 and 5, among others. It could be shown significantly that A549 cells, which were pre-incubated with ectoine, showed less or no bound viral Cov2 S1 protein on the cell surface. This clear and strong effect of ectoine is surprising and reproducible. It is also shown that the effect depends on the concentration of ectoine. It is thus surprisingly shown that compatible solutes have a significant protective effect on the host cells of ss(+) RNA viruses, depending on their concentration. This effect of solutes according to the invention enables prevention and treatment within the meaning of the present invention.

The observed effect of ectoine is currently based on a theory that is attributed to the protein and membrane stabilizing effect known for ectoine. It is assumed that compatible solutes according to the invention accumulate around the respective binding partner and shield them with a hydration shell. Without being limited to this theory, it is assumed that the hydration shell of the solutes, which presumably accumulate around the viral peplomers (spike proteins), stabilize the conformation of the folded or “closed” protein and in this way prevent the unfolding or “Opening” of the spike protein and thereby reducing or preventing accessibility of the receptor binding domain. As a result, the receptor-binding domain of the Cov2 S1 protein and the “viral receptor” of the host cell do not get in close enough proximity to prevent binding.

A similar effect can also be assumed on the effective side of the host cell. Here the solute according to the invention, preferably ectoine and/or its derivatives, appears to accumulate around the viral receptors. This achieves a double shielding of the receptor binding domain on the virus and the viral receptor on the host cell, which leads to a reduction in the binding between these binding partners. It cannot be excluded that the same applies to the cellular serine protease TMPRSS2, which is required for processing the SARS-CoV-2 spike protein to initiate fusion (Hoffmann et al. 2020).

Therefore, in a particular embodiment of the use according to the invention of at least one compatible solute or solute mixture, preferably ectoine and/or its derivatives, preferably contained in a composition according to the invention, the membrane of the transitional epithelia, the internal epithelial tissue and/or the endothelium is shielded.

The surface structures on human cells (viral receptors) required by the ss(+)-RNA viruses according to the invention as binding domains are preferably shielded. More preferably the binding domains of the viral receptors, comprising the angiotensin-converting enzyme 2 (ACE2), the aminopeptidase N (APN) and/or the dipeptidyl peptidase 4 (DPP4), are shielded by the at least one compatible solute or solute mixture, preferably ectoine and/or its derivatives. The angiotensin-converting enzyme 2 (ACE2) receptor is expressed in many organs, including the lungs, heart, liver, intestines, eyes and kidneys, and is used there by the viruses mentioned in the context of the invention as a receptor for binding to the respective cell.

Without being bound to it, one thesis on the mechanism of action is that by shielding membrane-bound surface proteins on the host cell, which the virus uses as a receptor to achieve binding to the human cell (“viral receptor”), binding of the virus and thus a viral infection of the cell is reduced or prevented. In particular, the sugar residues of the “viral receptor” required for the virus to bind are shielded. One thesis on the mechanism of action is that the shielding is achieved by the formation of hydrogen bridges between the OH groups of the viral receptors on the host cell and the solutes according to the invention, preferably ectoine. The strength water-binding capacity for ectoine is known, so it seems reasonable that the solutes attach and consequently recruit water molecules to form at least one or more hydration shells. This would mean that due to a lack of interaction between the binding partners, there would be no binding and thus ultimately no fusion of the membranes. Prevention of the above steps, based on this theory, would, according to the present invention, result in prevention or reduction of replication of the virus in the host cell. In this way, the course of the viral diseases within the meaning of the invention, including infections and/or inflammations of the transitional epithelium, internal epithelium and the endothelium, would be stopped and/or alleviated. This is used to treat a SARS-CoV-2 infection or to prevent the typical symptoms from breaking out. In this way, the Covid-19 endotheliitis is preferably treated or preventively prevented.

Therefore, in a particular embodiment of the use of the compatible solute or solute mixture according to the invention, the membrane of the transitional epithelium, the internal epithelial tissue and/or the endothelium is shielded with compatible solutes.

In a further embodiment of the use according to the invention of the at least one compatible solute or solute mixture, preferably ectoine and/or its derivatives, the at least one solute or solute mixture is administered in combination with antiviral compounds, anti-inflammatory compounds, interleukin blockers, anti-inflammatory anti-cytokines, Inhibitors of viral receptors and/or vaccines.

The group of the aforementioned anti-viral compounds comprises antibodies and neutralizing antibodies selected from the group consisting of casirivimab, imdevimab, bamlanivimab, etesevimab, VIR-7831, VIR-7832, tocilizumab, sarilumab, adalimumab, siltuximab, BTN162b2 (BioNtech/Pfizer), an ACE2-Fc fused to immunoglobulin and CR3022. Other anti-cytokine therapies considered for use in treating or preventing diseases caused by ss(+)RNA viruses include etanercept, infliximab, golimumab, and certolizumab pegol.

The group of anti-viral compounds (antivirals) comprises antivirals that are also used against other pathogens of the Coronavriridae family comprising the SARS-CoV-1, SARS-CoV-2, MERS-CoV, HCoV-HKU1, HCoV-OC43, HCoV-NL63 and/or HCoV-229E viruses. Known antivirals which can be combined with ectoine and/or one of its derivatives are selected from favipiravir, lopinavir, ritonavir, acyclovir, ganciclovir, ribavirin, foscavir (foscarnet), penciclovir, alisporivir, remdesivir, molnupiravir (4482/EI DD-2801), ivermectin, umifenovir, oseltamivir, ASCO9F and galidesivir. The antiviral is preferably selected from acyclovir, ganciclovir, ribavirin, foscavir (foscarnet), remdesivir, molnupiravir (4482/EI DD-2801) and ivermectin.

The group of anti-inflammatory compounds comprises paracetamol, ibuprofen, immunoglobulin, IL-1 blockers, IL-6 inhibitors, tumor necrosis factors (e.g. adalimumab).

The group of inhibitors comprises helicase inhibitors, complementary inhibitors (rheumatoid drugs), protease inhibitors, indinavir (Crixivan), nelfinavir (Viracept), saquinavir (Fortovase), renin-angiotensin system (RAS) inhibitors, neuraminidase inhibitors (SARS virus), oseltamivir (Tamiflu), zanamivir (Relenza), reverse transcriptase inhibitors (SARS virus), lamivudine (Epivir) and zidovudine (Retrovir). Preferably, ectoine and/or one of the derivatives described here is combined with neuraminidase inhibitors or reverse transcriptase inhibitors, oseltamivir, zanamivir, lamivudine and zidovudine. Particularly preferably, ectoine and/or one of the derivatives described here is combined with neuraminidase inhibitors or reverse transcriptase inhibitors.

Another group are the Janus kinase inhibitors (JKI), which comprises baricitinib+remdesivir, ruxolitinib, tofacitinib and fedratinib. Other JKIs are the approved preparations oclacitinib, peficitinib, upadacitinib and filgotinib as well as the not yet approved preparations cerdulatinib, lestaurtinib, gandotinib, momelotinib, pacritinib, abrocitinib and deucravacitinib. Another group are Bruton's tyrosine kinase inhibitors, which include acalabrutinib, ibrutinib, and zanubrutinib, as well as the drugs spebrutinib, fenebrutinib, HM71224, ABBV-105, and ONO-4059, which are still in development.

The group of interferons comprises interferon α-2a (Roferon), interferon α-2b (Intron A), interferon α-nl (Wellferon), interferon α-n3 (Alferon), interferon β-1a (Rebif) and interferon β-1 b (Betaferon). The group of interleukin blockers (immunosuppressors/immune depressants) comprises dexamethasone, hydrocortisone, colchicine, fluvoxamine, anakinra (interleukin (IL)-1 inhibitor) and interferon beta.

A combination according to the invention enables a combination therapy, e.g. acute treatment to protect the tissue that is still intact, to support the rehabilitation of tissue that has already been attacked, to reduce infectious breath/sputum, each in combination with an immunizing therapy with a vaccine.

The combination of ectoine and one of the anti-viral compounds, anti-inflammatory compounds, interleukin blockers, anti-inflammatory anti-cytokines, inhibitors of the viral receptors and/or vaccines described here can take place simultaneously, successively or also at different times. Thus, a combination therapy comprises two of the different compounds or Active ingredients without being limited to simultaneous administration. In one embodiment, the respective combination in a preparation is present as a mixture of a compound described herein and ectoine and/or one of the derivatives described herein, or as a combination of separate preparations. A separate preparation includes any spatial separation of the selected combination such that mixing of ectoine and the selected compound is prevented during storage.

One embodiment is a preparation in the form of a two-chamber system, in which ectoine and/or one of the derivatives described here is present in one chamber and one of the compounds described here is present in the second chamber. The two-chamber system preferably comprises an inhalable formulation in each case. An inhalable formulation is therefore to be understood as being suitable for taking up the active ingredient(s) into the lungs using suitable aids. This embodiment is particularly preferably suitable for use in a combination therapy for the treatment or prevention of diseases caused by ss(+)RNA viruses of the family Coronavriridae, preferably SARS-Cov-2, the combination being administered to the patient as an inhalant (see e.g. FIG. 5). This formulation can be administered via the lungs with an inhaler (PMDI or DPI) or nebulizer (mechanical or electric or pneumatic).

Alternative embodiments for inhalation as part of a combination therapy include separate chambers or cartridges that are alternately administered and inhaled according to the instructions. Other routes of administration and formulations suitable for combination therapy in accordance with this invention are shown in FIG. 5. Thus, other embodiments of combination therapy include an inhalant containing ectoine and/or any of the derivatives described herein and another formulation of any of the anti-viral compounds, anti-inflammatory compounds, interleukin blockers, anti-inflammatory anti-cytokines, inhibitors of viral receptors and/or described herein vaccines.

For example, ectoine and/or any of the derivatives described herein may be provided with interferon beta as an inhalant. The inhalant can be a mixture of the preparations mentioned or these are present separately in a two-chamber system or in separate cartridges. In the first case, both preparations are inhaled through inhalation in parallel or alternately from the two-chamber system. If the cartridges are separate, it may be necessary to combine the cartridges alternately with a suitable tool and use them accordingly.

Combination therapy also includes ectoine and/or any of the derivatives described herein in the form of a nasal, oral and/or throat spray and any of the compounds described herein.

Another embodiment of a combination is the use of at least one solute or solute mixture according to the invention, preferably ectoine, with a corticoid preparation. This combination is preferably used for the treatment of viral diseases according to the invention of the lower airways, especially the lungs, particularly preferably in the form of an inhalant according to the invention.

In a further embodiment of the use of the compatible solute or solute mixture according to the invention, the at least one solute or the solute mixture, preferably ectoine and/or its derivatives, preferably contained in a composition according to the invention, is used in patients who come from regions with high fine dust pollution, belong to a professional group with high level of fine dust pollution, have a previous vascular disease and/or chronic respiratory diseases.

Special patient groups patients at risk (Wang et al 2020) are patients with weakened endothelial function, e.g. patients with diabetes, heart failure, hypertension, coronary artery disease, cardiovascular impairment, peripheral vascular disease, cerebrovascular disease, cerebrovascular insufficiency, immune deficiency and/or chronic obstructive pulmonary disease (COPD).

With regard to previous illnesses of the respiratory tract, smokers and people who are particularly exposed to fine dust pollution, asthmatics, etc. also belong to the risk group (Zhao et al. 2020). In particular, it has been shown that the virus leads to a more severe course of infection in people exposed to particulate matter exposure than in people with less exposure (Xiao Wu et al 2020).

A further object is a composition for use in the prevention or treatment of diseases caused by ss(+)RNA viruses of the family Coronavriridae, wherein at least one compatible solute or solute mixture as defined herein invention is included. The composition preferably contains a solute selected from the group consisting of glyceryl glucoside (Glycoin), mannosylglycerate (Firoin), mannosylglyceramide (Firoin-A), ectoine and compounds of the formula I and/or II. Ectoine, hydroxyectoine and glycoin are particularly preferred.

In a further embodiment of the composition according to the invention containing at least one solute or solute mixture, the at least one compatible solute or solute mixture is present in the composition in an amount of greater than or equal to 0.0001% by weight to less than or equal to 70% by weight, based on the total content of the composition. Further embodiments of the composition according to the invention contain greater than or equal to 0.001% by weight, greater than or equal to 0.01% by weight, greater than or equal to 0.1% by weight, greater than or equal to 1.0% by weight, greater than or equal to 1.5% by weight, greater than or equal to 2.0% by weight, greater than or equal to 2.5% by weight, greater than or equal to 3.0% by weight in each case less than or equal to 65% by weight, less than or equal to 60% by weight, less than or equal 55% by weight, less than or equal 50% by weight, less than or equal 45% by, less than or equal 40% by weight, less than or equal 35% by weight, less than or equal 30% by weight, less than or equal to 25% by weight, less than or equal to 20% by weight.

Preferred proportions of the solute according to the invention in the composition depend on the particular formulation. A composition according to the invention preferably contains at least one compatible solute or the solute mixture, preferably ectoine and/or its derivatives, in a proportion of greater than or equal to 0.5% by weight to less than or equal to 15% by weight, preferably greater than or equal to 0.5% by weight % to less than or equal to 10% by weight, greater than or equal to 0.5% by weight to less than or equal to 5.0% by weight, particularly preferably greater than or equal to 0.5% by weight to less than or equal to 4.0% by weight, based on the total content of the composition. The above ranges preferably apply to an infusion solution within the meaning of the present invention.

An embodiment of the inhalant solution according to the invention contains a proportion of greater than or equal to 2% by weight to less than or equal to 25% by weight, greater than or equal to 5% by weight to less than or equal to 25% by weight, preferably greater than or equal to 8% by weight to less than or equal to 22% by weight, greater than or equal to 10% by weight to less than or equal to 20% by weight and preferably greater than or equal to 10% by weight to less than or equal to 18% by weight.

In another embodiment of the inhalant solution according to the invention, lower concentrations of the at least one solute or solute mixture according to the invention, preferably ectoine and/or its derivatives, are used. In this embodiment, multiple applications of the content are preferred in order to achieve a dose that is comparably high with a lower concentration than with a single application. Which concentration the inhalant should have depends on the patient's condition, the desired therapy, any previous damage to the lungs and/or other criteria such as e.g. age and general constitution of the patient, which can affect the process of inhalation itself.

The use of an inhalant with a lower proportion of the at least one solute according to the invention makes sense particularly in the case of an already damaged lung which has a lower absorption capacity in comparison to a healthy lung.

Another embodiment of an inhalant solution according to the invention therefore has a proportion of greater than or equal to 0.5% by weight to less than or equal to 20% by weight, preferably greater than or equal to 1.3% by weight to less than or equal to 18% by weight, preferably greater equal to 2% by weight to less than or equal to 15% by weight and particularly preferably greater than or equal to 3% by weight to less than or equal to 10% by weight.

In a particular embodiment of the use of the invention the composition is in

i) solid forms including powder, granules, capsules, lozenges and effervescent tablets,

ii) liquid forms including solution, injection, infusion and suspension, and/or

iii) as a mixture including spray, aerosols and inhalant.

The composition of the present invention may be administered topically (e.g. to the eye), orally, nasally, intravenously, inhalatively, intratracheally and/or buccally (e.g. lozenge). The composition according to the invention is preferably administered orally, intratracheally or by inhalation. It can be a dry inhalant or an aerosol. Solutions are preferably used for the mouth, nose, throat and eyes in the form of drops or sprays. A combination is very possible depending on the required therapy.

In a particular embodiment of the use of the invention of the composition it is in liquid form as an infusion solution or in liquid form suitable for use with a nebulizer and/or respirator.

For a maximally saturated solute solution, concentrations that are just below the solubility limit of the respective solute are preferred. Such a highly concentrated solute solution can be used as a starting solution to prepare the solution required on site. In this way, individual solutions can be diluted depending on the administration required, the patient's condition (existing conditions, age, etc.) and/or desired therapy. This is especially beneficial when not differently concentrated solutions can be stored. A maximum solubility of about 620 g/L at 40° C. and about 550 g/L at 25° C. was determined for ectoine.

A solution for infusion is preferably administered at about body temperature. An average infusion bag has a volume of 500 ml. Based on a single administration of the full volume of an infusion bag, the infusion solution has a maximum concentration of 4% (isotonic) so that the body does not experience osmotic shock. Repeated administration is possible, but depends on the doctors decision.

The composition according to the invention is preferably used in the prevention or treatment of diseases caused by ss(+)RNA viruses of the family Coronavriridae, with an infusion being administered in combination with an inhalant.

In particular, a medical product (e.g. FIG. 6), which can be self-administered in between is the subject of the present invention. It is well known that healthcare workers are particularly at risk. This includes doctors and nurses in hospitals and staff in other facilities such as retirement homes, fire brigades, kindergartens and schools. The risk can be reduced by using a nasal spray, eye drop, lozenge, mouthwash and/or mouth spray according to the invention. This significantly reduces the risk of droplet infection and protects the people caring for and attending person.

Possible products, in particular medical products, can be subdivided according to the following applications: lungs, nose, mouth/throat and eyes, as shown in FIG. 6. Each of the applications shown in FIG. 6 can be combined with the at least one solute according to the invention or a solute mixture. FIG. 6 does not represent an exhaustive list, but only the most frequently used products. According to the invention, these are suitable for use in the prevention and treatment of diseases caused by ss(+) RNA viruses.

Another object of the present invention is a kit comprising
at least one ready-to-use composition according to the invention for inhalation, preferably in ampoules or disposable cartridges, and
a device, preferably for immediate and controlled, administration of the composition, in particular by inhalation.

A further object of the present invention is the use of a composition according to the invention in a device for the controlled delivery of the composition, wherein
the device being suitable for generating aerosols of the composition,
allowing the composition to be inhaled via the mouth and/or nose,
ensures a metered delivery of a defined spray burst of a liquid or dry composition,
ensures a metered delivery of a liquid composition into the eyes, and/or
allows the composition to be sprayed in the oral cavity, throat and/or nasal cavity.

An aerosol particle size VMD (Volumetric Median Diameter) in a range from greater than or equal to 3 μm to less than or equal to 7 μm is preferably achieved by the device, preferably electronically controlled, as a result of which improved inhalation is achieved. Particular preference is given to particle sizes in a range from greater than or equal to 3.0 μm to less than or equal to 6.5 μm, greater than or equal to 3.0 μm to less than or equal to 6.0 μm, greater than or equal to 3.0 μm to less than or equal 5.5 μm, greater than or equal to 3.0 μm to less than or equal to 5.0 μm, greater than or equal to 3.0 μm to less than or equal to 4.5 μm, greater than or equal to 3.5 μm to less than or equal to 5.0 μm, greater than or equal to 4.0 μm to less than or equal to 5.0 μm, each with a standard deviation of +/−0.005-0.1 μm.

In one embodiment of the aforementioned device, a continuous rate of 0.5 mL ectoine/min is delivered. This results in a delivery of 20 mg/min of ectoine, starting from a 1.3% ectoine inhalant solution.

### DESCRIPTION OF THE FIGURES

FIG. 1 Gating strategy to exclude the dead cells from the analysis. A) FL1 shows the green fluorescence resulting from the detection of cell-bound SARS-Cov-2 S1. Dead cells were counterstained with propidium iodide. Dead cell staining is visible by a shift in the FL2 axis. Thus, cells in gate R2 are live cells. B) The cells in gate R2 were plotted as a histogram and the mean fluorescence intensity of these cells was determined.

FIG. 2 Proof of the binding of the Cov2 S1 protein to A549 cells using flow cytometry. The mean fluorescence intensity of living cells is shown.

FIG. 3A Proof of the effect of ectoine compared to NADA on the binding of the Cov2 S1 protein to A549 cells. Cov2 S1 protein binding to A549 cells were measured using immunofluorescence and subsequent flow cytometry. The mean fluorescence intensity of living cells is shown. Measurement series n=2 for 1.25% and 2.5%, n=1 for 5%; the mean values from both measurements are shown.

FIG. 3B Depiction of the influence of ectoine on the binding of the Cov2 S1 protein to A549 cells (as in FIG. 3A), with the concentration range being extended to 0.25% to 7.5% ectoine. The mean values from both measurements are shown.

FIG. 4A Depiction shows the results as a relative increase in fluorescence. The increase in fluorescence of A549 cells due to the binding of the Cov2 S1 protein to the cell surface was calculated by dividing the mean fluorescence intensity of the protein-stimulated cells by the intensity of the non-protein-stimulated cells.

FIG. 4B The data of all experiments were combined, the background fluorescence eliminated and plotted against the concentration of the Cov2 S1 protein. The inhibitory effect of ectoine is significant even when the standard deviation is taken into account. In comparison, cells preincubated with NADA are comparable to untreated cells

FIG. 5 This figure shows all possible formulations and applications of the solutes according to the invention, or compositions containing at least one solute or solute mixture, preferably ectoine and/or its derivatives, summarized. Only the infusion solution is not shown in this figure.

### REFERENCES

Costa et al. da Costa M S, Santos H, Galinski E A. An overview of the role and diversity of compatible solutes in Bacteria and Archaea. Adv Biochem Eng Biotechnol. 1998; 61:117-53.
Dong et al. Dong, J.; Xu, X.; Liang, Y.; Head, R.; Bennett, L. Inhibition of angiotensin converting enzyme (ACE) activity by polyphenols from tea (Camellia sinensis) and links to processing method. Food Funct. 2011, 2, 310-319.
Fang Li Structure, Function, and Evolution of Coronavirus Spike Proteins. Annu. Rev. Virol. 2016. 3:237-61. doi: 10.1146/annurev-virology-110615-042301
Hahn et al. Marc Benjamin Hahn, Frank Uhlig, Tihomir Solomun, Jens Smiatek and Heinz Sturmad Combined influence of ectoine and salt: spectroscopic and numerical evidence for compensating effects on aqueous solutions Phys. Chem. Chem. Phys., 2016, 18, 28398
Hoffmann et al. Markus Hoffmann, Hannah Kleine-Weber, Simon Schroeder, . . . , Marcel A. Müller, Christian Drosten, Stefan Pöhlmann SARS-CoV-2 Cell Entry Depends on ACE2 and TMPRSS2 and Is Blocked by a Clinically Proven Protease Inhibitor Cell 181, 271-280 Apr. 16, 2020 Elsevier Inc. https://doi.org/10.1016/j.cell.2020.02.052
Roychoudhury et al. Roychoudhury A, Haussinger D, Oesterhelt F. Effect of the compatible solute ectoine on the stability of the membrane proteins. Protein Pept Lett. 2012 August; 19(8):791-4.
Tay et al. Matthew Zirui Tay, Chek Meng Poh, Laurent Rénia, Paul A. MacAry and Lisa F. P. Ng The trinity of COVID-19: immunity, inflammation and intervention. Nature Reviews, Immunology doi: 10.1038/s41577-020-0311-8
Tortoricia M. Alejandra Tortoricia David Veeslera, Structural insights into coronavirus entry. Advances in Virus Research, Volume 105. 2019. doi.org/10.1016/bs.aivir.2019.08.002
Uhal et al. Bruce D. Uhal, MyTrang Dang, Vinh Dang, Roger Llatos, Esteban Cano, Amal Abdul-Hafez, Jonathan Markey, Christopher C. Piasecki and Maria Molina-Molina Cell cycle dependence of ACE-2 explains downregulation in idiopathic pulmonary fibrosis Eur Respir J 2013; 42: 198-210 doi: 10.1183/09031936.00015612
Wang et al. Bolin Wang, Ruobao Li, Zhong Lu, Yan Huang Does comorbidity increase the risk of patients with COVID-19: evidence from meta-analysis. AGING 2020, Vol. 12, No. 7
Wu et al. Xiao Wu M S, Rachel C. Nethery PhD, M. Benjamin Sabath M A, Danielle Braun PhD, Francesca Dominici PhD. Exposure to air pollution and COVID-19 mortality in the United States preprint doi: 10.1101/2020.04.05.20054502
Zhao et al. Qianwen Zhao, Meng, Rahul Kumar, Yinlian Wu, Jiaofeng Huang, Ningfang Lian, Yunlei Deng, Su Lin. The impact of COPD and smoking history on the severity of Covid-19: A systemic review and meta-analysis. Accepted article

The following examples show the efficacy of selected compatible solutes to demonstrate the feasibility of the invention, but without limiting the invention to them.

### EXAMPLES

### Example 1: Inhibition of Binding of the SARS-CoV-2 Spike S1 Protein to A549 Cells by Compatible Solutes

Material

Ectoine: (4S)-2-Methyl-1,4,5,6-tetrahydropyrimidine-4-carbon acid, CAS Nr. 96702-03-3

NADA: Nγ-Acetyl-L-2,4-diaminobutyric acid, CAS Nr. 1190-46-1

Hydroxyectoine: CAS-Nr. 165542-15-4

Glycoin: CAS-Nr. 22160-26-5

Mannosylglycerate: CAS-Nr. 164324-35-0

Glycine-Betaine: CAS Nr. 107-43-7

L-Prolin: CAS-Nr. 147-85-3

SARS-CoV-2 Spike S1 protein: trenzyme life science services, Cat. No. P2020-001, Lot No.: 11PO

Method

Angiotensin-converting enzyme 2 (ACE2)-expressing A549 cells from the American Type Culture Collection (ATCC) were used (Uhal et al. 2013). The adherent cells were routinely cultured in T-75 flasks in DMEM with 10% FCS containing penicillin and streptomycin as antibiotics in a CO₂ incubator (5% CO₂, 37° C.). When the bottom of the flask was 2/3 covered with cells, they were detached with accutase.

To perform the assay, the A549 cells were cultured to confluence in DMEM containing 10% FCS, and confluent culture was continued for another four days during which time the cell culture medium was replaced.

For the inhibition experiment, the cells were detached with accutase. Each 105 cells were transferred to a sample tube and pretreated with different concentrations of the compatible solute (Table 2) to give a final volume of 100 μl.

After 30 min incubation, the HIS-tagged SARS-Cov2-S1 protein is added to each tube and incubated for an additional 60 min on a tumble shaker at 450 rpm and room temperature.

TABLE 3
compatible Solute + CoV2 S1

| Concentration [%] | Amount of CoV2 | | | |
|---|---|---|---|---|
| compatible Solute | S1 [μg] | | | |
| 7.5 | 5 | 2 | 1 | 0 |
| 5 | 5 | 2 | 1 | 0 |
| 2.5 | 5 | 2 | 1 | 0 |
| 1.25 | 5 | 2 | 1 | 0 |
| 0.75 | 5 | 2 | 1 | 0 |
| 0.25 | 5 | 2 | 1 | 0 |
| 0 | 5 | 2 | 1 | 0 |

The evaluation was carried out via immunofluorescence using flow cytometry. Binding of the SARS-CoV-2 Spike 51 protein is analyzed by indirect immunofluorescence using a CyFlow SL (Sysmex GmbH) flow cytometer. The cells were first centrifuged at 300×g, 5 min and then resuspended in 100 μl PBS with 1.5% fetal calf serum and 10 mM sodium azide. The cells were then challenged for 30 minutes with 5 μg/ml mouse antibody directed to the HIS tag (Biolegend). After washing (PBS with 1.5% fetal calf serum and 10 mM sodium azide), the cells were labeled with the secondary antibody (20 μg/ml, rabbit anti-mouse IgG (H+L), Alexafluor 488, Invitrogen) incubated for 20 minutes. The cells were then washed once and resuspended in 1 ml PBS without azide/FCS. Shortly before the measurement, 10 μl of a 0.5 mg/ml propidium iodide solution were added. Thereafter, the cells were immediately filtered through a 50 μm cell strainer and then immediately measured. All steps are performed at 4° C. in the dark.

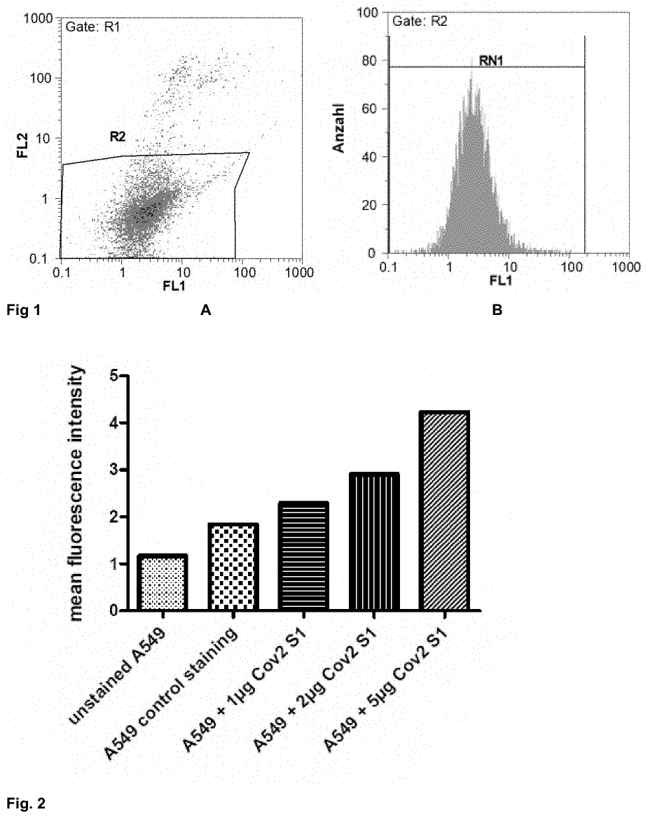

The fluorescence of the A549 cells is then analyzed by flow cytometry. For this purpose, a region is placed on the live cells (the dead cells can be excluded from the analysis since they absorb the red propidium iodide dye) and the geometric mean relative fluorescence (MFI) of the cells in this region is determined as in FIG. 1 shown.

### Example 2: Proof of the Binding of the SARS-Cov2-S1 Protein as a Function of the Concentration in A549 Cells

A549 cells are grown to confluency in DMEM with 10% FCS. Thereafter, the cells were supplied with fresh cell culture medium every day. Subsequently, the cells were stimulated with different concentrations of the SARS-Cov2-S1 protein without an extremolyte, as described in example 1. The analysis was performed by cytometry.

TABLE 4
MFI-values of FIG. 2. The corresponding
coefficient of variation in percent (CV %) of the
fluorescence intensity of the cells is also given.

| | MFI | CV % |
|---|---|---|
| Unstained A549 | 1.17 | 71.39 |
| A549 control staining | 1.84 | 87.62 |
| A549 + 1μCov2 S1 | 2.29 | 65.24 |
| A549 + 2μCov2 S1 | 2.90 | 70.55 |
| A549 + 5μCov2 S1 | 4.23 | 88.75 |

In a comparison of unstained (“unstained A549”) and stained A549 control cells (“A549 control staining”), a slight background staining is measured. Control stained A549 cells are cells that were not stimulated with Cov2 S1 protein but treated with a Fitc-labeled antibody for staining. Taking these controls into account, the signal of the bound spike protein was already detectable after stimulating the cells with 1 μg Cov2 S1. Increased protein binding to the A549 cells was detectable by using higher amounts of the protein.

Thus, by means of this assay, a concentration-dependent binding of a binding domain, such as Cov2 S1, to ACE-expressing cells, such as A549, can be detected and a substance can be examined for its effect of inhibiting the binding of Cov2 S1 to A549 cells.

The assay can be carried out in a modified form using the cell lines mentioned below. Any cell line which expresses angiotensin-converting enzyme 2 (ACE2), aminopeptidase N (APN) and/or dipeptidyl peptidase 4 (DPP4) is a suitable cell line for the purposes of the assay according to the invention. The detailed properties of the cell lines listed below can be looked up, inter alia, in the protein atlas: https://www.proteinatlas.org/ENSG0000013Q234-ACE2/cell and are known to the person skilled in the art.

TABLE 5
ACE-Receptor expressing cell lines

| Name | ACE2 | Origin | Provider (Availability examples) |
|---|---|---|---|
| COS7 | + | Kidney | Creative biogene CSC-RO0290, |
| | | (monkey) | LGCStandards ATCC ® CRL-1651 ™ |
| Hek293 | + | human | Creative biogene CSC-RO0292/ |
| | | kidney | LgcStandards ATCC ® CRL-1573 ™ |
| Hek293T | + | human | Creative biogene CSC-RO0641 |
| | | kidney | LgcStandards ATCC ® CRL-11268 ™ |
| Hep2G | + | Liver | LgcStandards ATCC ® HB-8065 ™ |
| HUVEC | + | Endothelium | LgcStandards ATCC ® CRL-1730 ™ |
| RPMI-8226 | + | Peripheral | LgcStandards ATCC ® CCL-155 ™ |
| | | blood | |

A suitable antibody which detects the expression of the receptor on the cell surface is preferably included in the assay. Cell lines that show low mRNA expression for ACE are cultivated in DMEM with at least 10% FCS. Hep 2G cells show high expression of the RNA for ACE. HUVEC cells express ACE2 and are cultured in Vascular Cell Basal Medium using the Endothelial Cell Growth Kit (ATCC). As a control, MRC5 cells that do not express the ACE receptor are included. In addition, Hoffmann et al. 2020 demonstrated that the cells are not infected by SARS CoV2. Therefore, the MRC5 cells represent an excellent control for the specificity of the binding assay used. MRC5 cells are cultured in DMEM with 10% FCS.

The HUVEC cell line, which is already used for ACE inhibition tests (Don et al.), is particularly suitable for providing evidence of a potential Covid-19 endotheliitis as currently discussed and the positive effect of compatible solutes according to the invention, such as the ectoine.

In the binding experiment, the aforementioned cell lines are incubated with the CoV2 S1 concentrations shown in Table 6.

### Example 3: Influence of Ectoine on the Binding of the SARS-Cov2-S1 Protein to A549 Cells

TABLE 6
compatible Solute + CoV2 S1

| Concentration [%] | Amount of | | | |
|---|---|---|---|---|
| compatible Solute | CoV2 S1 [μg] | | | |
| 20 | 5 | 2 | 1 | 0 |
| 15 | 5 | 2 | 1 | 0 |
| 10 | 5 | 2 | 1 | 0 |
| 5 | 5 | 2 | 1 | 0 |
| 2.5 | 5 | 2 | 1 | 0 |
| 1.25 | 5 | 2 | 1 | 0 |
| 0 | 5 | 2 | 1 | 0 |

In order to investigate whether ectoine has an influence on the binding of the Cov2 S1 protein to A549 cells, detached cells were examined with different concentrations of a solute according to the invention, such as ectoine. For this purpose, the cells were pre-incubated with the respective solute and then treated with different concentrations of the Cov2 S1 protein (see example 1 and 2).

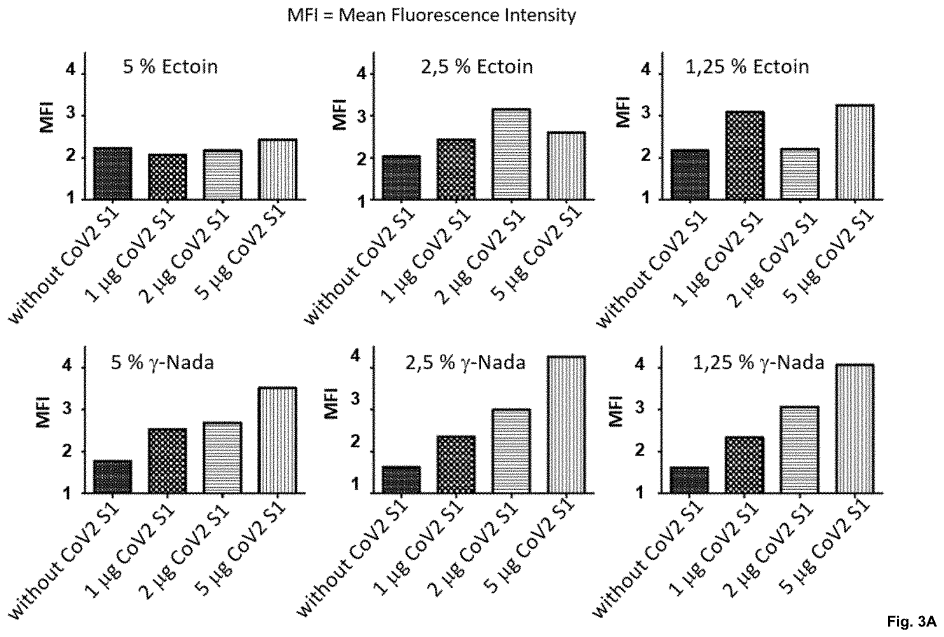

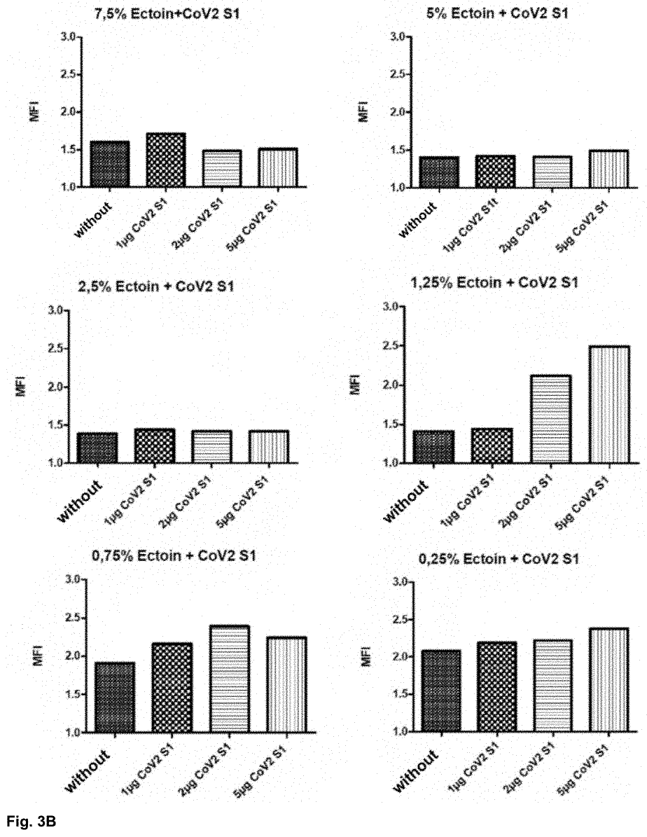

In this “proof-of-concept” experiment with n=1, a lower fluorescence of the Cov2 S1 protein bound to A549 cells was measured even in the presence of 2.5% ectoine (data not shown). This indicates reduced binding of Cov2 S1 protein to A549 cells in the presence of ectoine. No effect on the binding between Cov2 S1 and the A549 cells was found for NADA compared to ectoine. Both the experimental mixture [1.25/2.5% NADA+5 μg Cov2 S1+A549] and [5 μg Cov2 S1+A549] show the same mean fluorescence (cf. FIGS. 2 and 3). Thus, NADA does not appear to affect the binding of the virus to the host cell.

To verify the experiment, the concentrations of 1.25% and 2.5% were repeated and 5% of the respective solute was also examined. The results are shown in table 7.

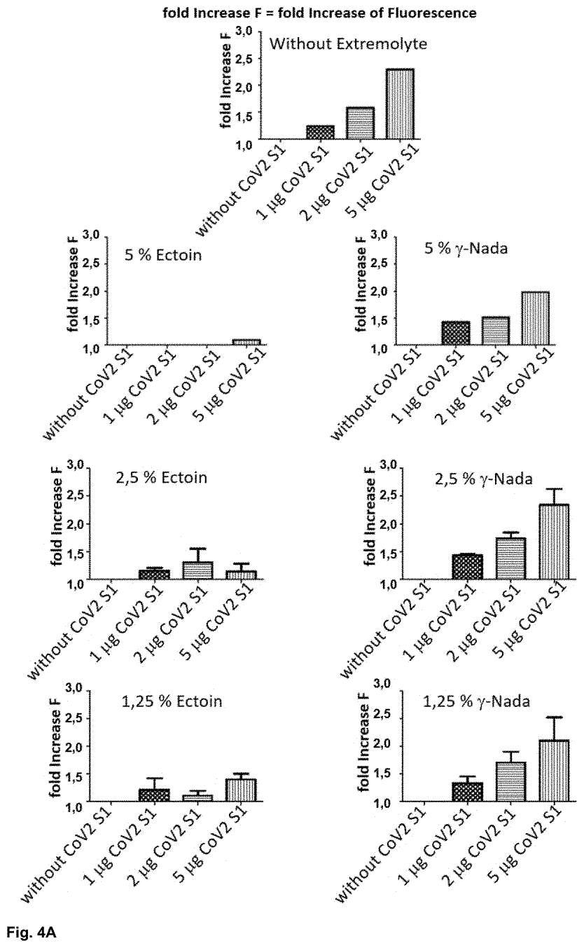

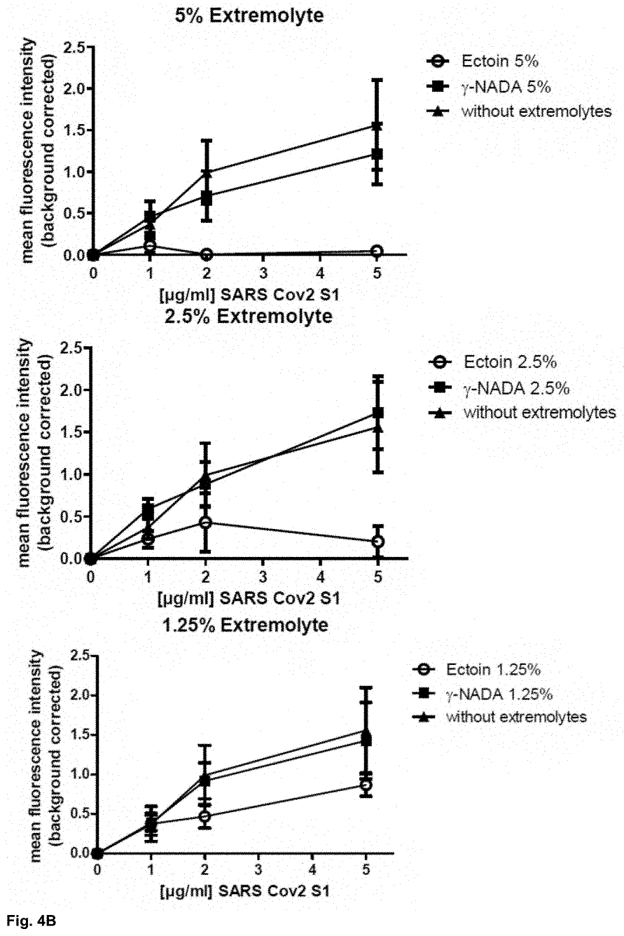

TABLE 7
MFI-values of FIG. 4. The corresponding
coefficient of variation in percent (CV %) of the
fluorescence intensity of the cells is also given.

| | MFI | CV % |
|---|---|---|
| 5% Ectoin | | |
| Without Cov2 S1 | 2.23 | 181.07 |
| 1μCov2 S1 | 2.07 | 88.77 |
| 2μCov2 S1 | 2.17 | 134.70 |
| 5μCov2 S1 | 2.43 | 123.09 |
| 2.5% Ectoin | | |
| Without Cov2 S1 | 2.03 | 131.63 |
| 1μCov2 S1 | 2.43 | 142.38 |
| 2μCov2 S1 | 3.15 | 174.07 |
| 5μCov2 S1 | 2.61 | 159.93 |
| 1.25% Ectoin | | |
| Without Cov2 S1 | 2.17 | 117.24 |
| 1μCov2 S1 | 3.09 | 151.13 |
| 2μCov2 S1 | 2.21 | 152.85 |
| 5μCov2 S1 | 3.25 | 144.79 |
| 5% γ-NADA | | |
| Without Cov2 S1 | 1.77 | 102.48 |
| 1μCov2 S1 | 2.52 | 113.55 |
| 2μCov2 S1 | 2.68 | 124.69 |
| 5μCov2 S1 | 3.51 | 121.95 |
| 2.5% γ-NADA | | |
| Without Cov2 S1 | 1.62 | 101.84 |
| 1μCov2 S1 | 2.35 | 129.73 |
| 2μCov2 S1 | 2.99 | 144.33 |
| 5μCov2 S1 | 4.26 | 130.39 |
| 1.25% γ-NADA | | |
| Without Cov2 S1 | 1.61 | 83.86 |
| 1μCov2 S1 | 2.33 | 126.42 |
| 2μCov2 S1 | 3.06 | 142.59 |
| 5μCov2 S1 | 4.06 | 154.67 |

With 5% ectoine, a complete inhibition of the binding of the Cov2 S1 protein to A549 cells could be shown. In comparison, no inhibition could be achieved with NADA. Here the mean fluorescence in the presence of 1.25%, 2.5% or 5% NADA increases comparably to the mean fluorescence of the test set without a compatible solute (cf. FIG. 2). The experiment with NADA and ectoine was repeated with the concentrations listed in Table 3. It was confirmed that NADA had no effect on Cov2 S1 protein binding to A549 cells (data not shown), whereas ectoine repeatedly had a significant effect (FIG. 3b). All tests carried out were summarized in order to determine the significance of the effect. The background fluorescence of the A549 cells was subtracted and the specific values plotted against the concentration of Cov2 S1 protein binding (FIG. 4b). It was confirmed that ectoine has a significant effect on A549 cells and inhibits the binding of the Cov2 S1 protein. Significance calculation:

| Mann Whitney test | |
|---|---|
| P value | 0.0286 |
| Exact or approximate P value? | Exact |
| P value summary | * |
| Significantly different (P < 0.05)? | Yes |
| One- or two-tailed P value? | Two-tailed |
| Sum of ranks in column Ectoin | 10.26 |
| %, NADA 5%, | |
| Mann-Whitney U | 0 |
| Difference between medians | |
| Median of column Ectoin 5% | 0.3050, n = 4 |
| Median of column NADA 5%, | 1.820, n = 4 |
| Difference: Actual | 1.515 |
| Difference: Hodges-Lehmann | 1.505 |

### Example 4: Influence of Glycoin and Ectoine on the Binding of the SARS-Cov2-S1-Proteins

Experiments analogous to Example 3 are carried out with the cell lines HUVEC, HEK293, RPMI-8226 and Hep G2 (Table 5). Ectoine and glycoin in different concentrations (Table 3) are tested as compatible solutes.

### Example 5: Influence of Glycoin, Mannosylglycerate, Hydroxyectoine and Ectoine on the Binding of the SARS-Cov2-S1 Protein

Experiments analogous to example 3 are carried out with the cell lines HUVEC, HEK293, RPMI-8226 and Hep G2 (Table 5), the Cells are not pre-incubated with ectoine, but ectoine or glycoin and Cov2 S1 protein are administered at the same time. Ectoine, hydroxyectoine, mannosylglycerate and glycoin are tested as solutes in various concentrations (Table 3).

As a further approach, the Cov2 S1 protein is pre-incubated with ectoine or glycine and then the cells are incubated with the pre-incubated mixture [Cov2 S1 protein+ectoine] in different concentrations according to Table 3. The pre-incubation [Cov2 S1 protein+ectoine] takes place for 5 min, 10 min and 30 min each at room temperature. The analysis then takes place analogously to example 3. The experiment is carried out with the cell lines HUVEC, HEK293, RPMI-8226 and Hep G2 (Table 5).

### Example 6: Calculation of the Increase in Relative Fluorescence

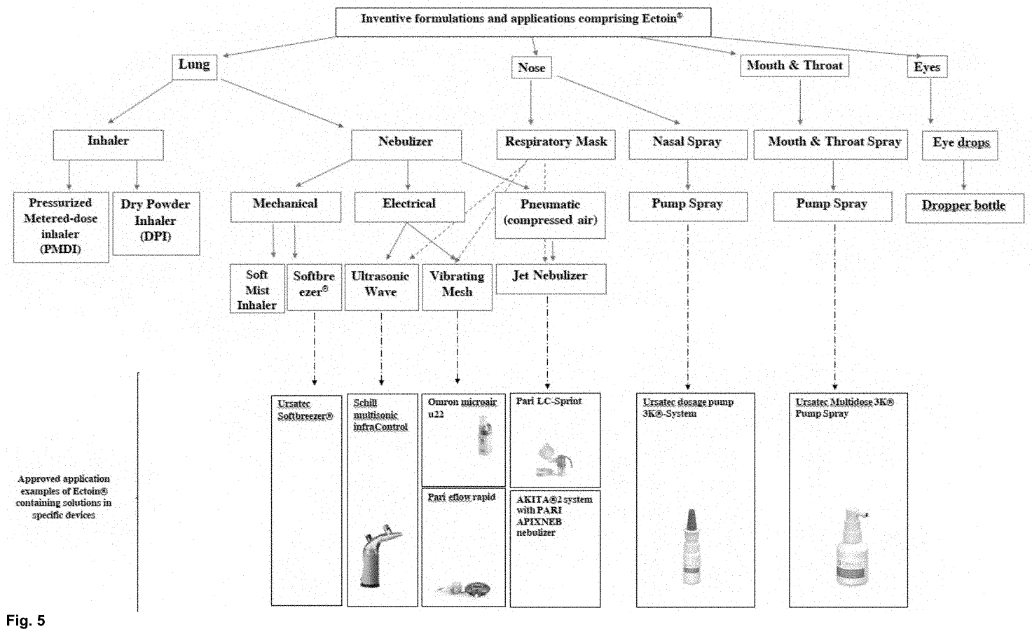

To make the experiments comparable, the increase in fluorescence of A549 cells due to the binding of the Cov2 S1 protein to the cell surface was calculated by dividing the mean fluorescence intensity of the cells by the mean fluorescence intensity of the background staining. To determine background staining, cells were stained with an antibody in the absence of Cov2 S1. The results are shown in FIG. 5.

It can be seen that in the absence of a compatible solute, a concentration-dependent increase in mean fluorescence (fold fluorescence) is detected upon addition of the Cov2 S1 protein to A549 cells. While ectoine already shows a positive effect at 1.25% and inhibits the binding of the Cov2 S1 protein to A549 cells, no significant effect can be demonstrated for NADA at the same concentration. The effect of ectoine increases with increasing concentration and achieves complete inhibition of binding at 5% ectoine. In comparison, little inhibition of binding is shown with 5% NADA.

### Example 7: Atomic Force Spectroscopy to Determine the Effect of Compatible Solutes on the Stability of Membranes

Based on the method of Roychoudhury et al. a method is carried out using atomic force microscopy to detect a bond between a viral membrane-bound protein, in particular peplomer, and a human membrane-bound surface protein. The procedure is based on the following steps.

The binding domain to be tested or the entire protein is bound to a surface. The surface can be a membrane and even a whole cell that expresses the receptor such as ACE2, APN and/or DPP4. At least two different approaches are tested, one approach contains the human viral receptor with a compatible solute (ectoine 1 M) in a buffer (300 mM KCl and 20 mM Tris at pH 7.8) and one approach contains the human viral receptor in the buffer without solute. In a next step, the tip of the atomically guideable arm (AFM tip) of the device (AFM device from Asylum Research, Olympus OMCL TR400 silicon nitride canilever with a spring constant of 20 pN/nm) is provided with a viral receptor binding domain, preferably S1, or the whole protein, preferably of SARS-CoV-2. The protein has an HIS tag. This tip is then approached to the bound sample (viral receptor with/without solute) and gradually brought into contact. While approaching as well as during moving away from the bound protein, the Newton force is versus the distance between the potential binding partners—here ACE2 and S1—and recorded as a force curve (retraction speed of 400 nm/s).

The measured power of the experimental approaches with solute compared to without solute allow conclusions to be drawn about molecular interactions between a membrane-bound protein, such as ACE2, and the potential binding partner, such as the SARS-CoV-2 protein, and statements about the shielding effect of the solute on membrane-bound proteins, as already described for ectoine in Roychoudhury et al. The method is suitable for testing all compatible solutes.

The atomic force spectroscopy is carried out with the following combinations as an example

Ectoine—Spike S1 protein

Hydroxyectoine—Spike S1 protein

Glycoin—Spike S1 protein

Mannosylglycerat—Spike S1 protein

### Example 8: Examples of Formulations of the Solute According to the Invention A-1) Solute-Containing Inhalant Solution 13%

The aim of an inhalant within the meaning of the invention is to wet the lung surface as well and as extensively as possible with a thin layer of ectoine hydrate. A theoretically maximally and completely wetted lung is calculated using the data from Hahn et al. and Roychoudhury et al.

The area of a hydrated ectoine: 3.5e10⁻¹⁰ m*3.5e10⁻¹⁰ m*3.14=3.85e10⁻¹⁹ m² (circle formula). Distance ectoine to a water molecule=0.35 nm

Assuming that this is equal to the radius and the surface area of the lungs=100-150 m², it is calculated that 2.6e10²⁰ ectoine molecules are required to achieve a monolayer hydration shell for a complete occupancy of the area. For two hydration shells, 5.2e10²⁰ ectoine molecules would be required

It should be noted that the above calculation applies to a rather small lung and the area can be up to 1.5 times larger. The assumed radius of hydrated ectoine at 0.35 nm is the closest possible state. A less dense state (r=0.8 nm) would require less ectoine. Therefore, the present calculation is only to be regarded as indicative and may need to be adjusted to the size and/or age of the patient in individual cases.

Molecular weight ectoine: 142.16 g/mol

1 mol=6.022e10²³ particles

Amount of ectoine=5.2e10²⁰/(6.022e10²³×mol-1)=0.864e10⁻³ mol=0.123 g ectoine

With a 13% solution (130 g/I) that would be 0.00094 l or 0.94 mL

A dose of between 1-1.5 mL of an inhalant solution containing 13% solute would therefore be required to effectively arrive in the lungs so that ectoine is theoretically distributed homogeneously in the lungs. This determined concentration is based on the assumption of a single application

A-2) Solute Inhalant Solution Containing 3.9%

Starting from an inhalant solution containing 3.9% solute, a three-time application, e.g. distributed throughout the day, necessary to achieve a dose comparable to the 13% solution.

A) Infusion Solution 4%

An isotonic NaCl solution of 0.9% has an osmolar activity of 286 mOsmol/kg H₂O. A 2% ectoine solution has an osmolarity of 147 mOsmol/kg H₂O. An isotonic ectoine solution thus has a concentration of 3.89%.

An isotonic infusion solution is preferred so as not to irritate or damage the tissue. Therefore, an isotonic ectoine infusion solution contains 3.89% ectoine. Another combination is an ectoine infusion solution in combination with a NaCl solution suitable for infusion. Such a product contains a small amount of NaCl in combination with an adapted ectoine solution, resulting in an isotonic ectoine-NaCl infusion solution.

With the other solutes within the meaning of the invention, corresponding infusion solutions can be calculated according to their osmolarity.

| Solute | Solute [%] | | | | NaCl [%] |
|---|---|---|---|---|---|
| Ectoine | 3.0 | 3:5 | 3.8 | 4 | 0.9++ |
| Hydroxyectoine | * | * | * | * | 0.9++ |
| Betaine | * | * | * | * | 0.9++ |
| Glycoin | * | * | * | * | 0.9++ |

*To be determined according to the osmolarity or ++ to be adapted to a physiologically compatible solution

In an i.v. pharmacokinetics study, an amount of ectoine of 100 mg/kg was used in the rat. Oral toxicity data indicate a NOAL of 2000 mg/kg body weight/day. Orally taken ectoine (1000 mg/kg) resulted in a plasma concentration of 99 μg/ml in rats.

1. A method of preventing or treating a disease caused by a ss(+)RNA virus of the Coronavriridae family, said method comprising administering to a patient in need thereof a composition comprising at least one compatible solute or solute mixture, wherein the at least one compatible solute or solute mixture is selected from organic and highly water soluble compounds.

2. The method according to claim 1, wherein the at least one solute has a water-binding capacity of greater than or equal to 7 mol/mol H₂O/solute.

3. The method according to claim 1, wherein the at least one solute is selected from glyceryl glucoside (Glycoin), glycine betaine, mannosylglycerate (Firoin), mannosylglyceramide (Firoin-A), ectoine and its derivatives of formula I and/or II and the physiologically compatible salts, amides and esters of the aforementioned compounds, wherein
in formula I and in formula II 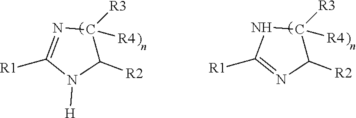
R1=H or alkyl,
R2=H, COOH, COO-alkyl or CO—NH—R5,
R3 and R4 are each independently H or OH,
R5=H, alkyl, an amino acid residue, dipeptide residue or tripeptide residue n=1, 2 or 3,
Alkyl=an alkyl radical with C1-C4 carbon atoms.

4. The method according to claim 1, wherein the disease is caused by an ss(+)RNA virus of the genus Betacoronavirus and/or Alphacoronavirus.

5. The method according to claim 1, to wherein the disease is caused by an ss(+)RNA virus selected from SARS-CoV-1, SARS-CoV-2, MERS-CoV, HCoV-HKU1, HCoV-OC43, HCoV-NL63 and/or HCoV-229E.

6. The method according to claim 1, wherein the disease comprises infection and/or inflammation of the transitional epithelial tissues and/or internal epithelial tissues.

7. The method according to claim 1, wherein the disease comprises infection and/or inflammation of the endothelium.

8. The method according to claim 1, wherein the ss(+)RNA virus interacts with at least one membrane-bound protein or component thereof on human cells and uses this protein or component thereof as a receptor for binding to the cell.

9. The method according to claim 1, wherein the ss(+)RNA virus interacts with a receptor selected from angiotensin converting enzyme 2 (ACE2), aminopeptidase N (APN) and/or dipeptidyl peptidase 4 (DPP4).

10. The method according to claim 1, wherein the at least one compatible solute reduces or prevents the unfolding and/or opening of the viral protein suitable for binding to the human receptor of the ss(+)RNA virus.

11. The method according to claim 1, wherein multiplication of the ss(+)RNA virus is reduced or prevented.

12. The method according to claim 1, wherein the at least one compatible solute shields the membrane of transitional epithelia, the internal epithelial tissues, and/or the endothelium.

13. The method according to claim 1, wherein the at least one solute is administered in combination with anti-viral compounds, anti-inflammatory compounds, interleukin blockers, anti-inflammatory anti-cytokines, inhibitors of viral receptors and/or vaccines.

14. The method according to claim 1, wherein the at least one solute is administered to a patient who comes from a region with high fine dust pollution, belongs to a professional group with high fine dust pollution, has a previous vascular disease and/or a chronic respiratory disease.

15. The method according to claim 1, wherein the at least one compatible solute or solute mixture is present in the composition in an amount of greater than or equal to 0.0001% by weight to less than or equal to 70% by weight based on a total content of the composition.

16. The method according to claim 15, wherein the composition is in
i) a solid form including powder, granules, capsules, lozenges and effervescent tablets,
ii) a liquid form including solution, injection, infusion and suspension, and/or
iii) as a mixture including spray, aerosols and inhalants.

17. The method according to claim 15, wherein the composition is in liquid form for solution for infusion or in liquid form suitable for use with a nebulizer and/or respirator.

18. The method according to claim 15, wherein an infusion is administered in combination with an inhalant.

19. A kit comprising
at least one ready-to-use composition according to claim 15 for inhalation, and
a device for administering the composition.

20. The method according to claim 15, wherein a device for the controlled delivery of the composition is used, wherein
the device is suitable for generating aerosols of the composition,
allows the composition to be inhaled via the mouth and/or nose,
ensures a metered delivery of a defined spray burst of a liquid or dry composition,
ensures a metered delivery of a liquid composition into the eyes, and/or
allows the composition to be sprayed in the oral cavity, throat and/or nasal cavity

21. A method of identifying a compatible solute according to claim 1, comprising the steps of
providing a cell line which has membrane-bound surface proteins as potentially viral receptors,
contacting the cells with a compound which is potentially a compatible solute according to claim 1,
adding a viral receptor binding domain comprising a measurable signal,
incubation of approach of the cells contacted with the potentially compatible solute and the viral receptor binding domain comprising a measurable signal,
recording the signal measurable on the cell and
determination of a reduced binding between the viral receptor binding domain and the human membrane-bound surface protein.
